(19) United States
(12) Patent Application Publication
(10) Pub. No.: US 2022/0331529 A1
(43) Pub. Date: Oct. 20, 2022

(54) DRUG DELIVERY DEVICE INCORPORATING ELECTRICAL SYSTEM CONTAMINATION PROTECTION, POWER SOURCE MANAGEMENT, POWER SOURCE MONITORING, AND/OR POWER SOURCE OPERATION

(71) Applicant: JANSSEN PHARMACEUTICALS, INC., Titusville, NJ (US)

(72) Inventors: Jason L. HARRIS, Lebanon, OH (US); Peter KRULEVITCH, Pleasanton, CA (US); Frederick E. SHELTON, IV, Cincinnati, OH (US); George SZABO, Hatfield, PA (US)

(21) Appl. No.: 17/763,590

(22) PCT Filed: Sep. 24, 2020
(86) PCT No.: PCT/IB2020/058968

§ 371 Date: Mar. 24, 2022

Related U.S. Application Data
(60) Provisional application No. 62/905,475, filed on Sep. 25, 2019.
(60) Provisional application No. 62/905,478, filed on Sep. 25, 2019.

(51) Int. Cl. A61M 15/00; A61M 5/20; A61M 5/142

(52) U.S. Cl. CPC A61M 15/0001; A61M 5/2033; A61M 5/14248; A61M 2205/0233; A61M 2205/0238; A61M 2205/3327; A61M 2205/3368; A61M 2205/3306; A61M 2205/3584; A61M 2205/3553; A61M 2205/8206; A61M 2205/8243; A61M 2205/18; A61M 2205/50

(57) ABSTRACT

The present disclosure relates to a drug administration device configured to administer a drug. In an exemplary embodiment the drug administration device includes a housing including first and second housing electrical contacts, a dispensing mechanism, a drug holder, a removable power supply including first and second power supply electrical contacts, and at least one protection mechanism in a first protecting configuration when the removable power supply is not received within the housing and in a second configuration when the removable power supply is received within the housing. In another exemplary embodiment the drug administration device includes a housing, a dispensing mechanism disposed within the housing, a removable power supply that is configured to be received within the housing, a sensor configured to determine remaining charge of the removable power supply, and a processor configured to modify operation of the drug administration device in response to data produced by the sensor.

### FIELD

The embodiments described herein relate generally to a device for administering a drug. The present disclosure further relates to a drug administration device with anti-contaminant protection, power source management, power source monitoring, and/or power source operation.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

Further, it is desirable for drug administration devices to be suitable for use in a variety of environments. Many environments in which a patient may need to use a drug administration device comprise moisture. Moisture contamination can cause malfunction of the electrical components of a drug administration device. Moisture contamination can also cause the drug administration device's dispensing mechanism to incorrectly administer the drug. Therefore, there is a need to protect a drug administration device from moisture contamination. It is also desirable to provide drug administration devices that will prevent a user from administering a contaminated drug.

Further, a drug administration device may operate but not have enough power to successfully administer a drug. This risks harming the patient.

### SUMMARY

In a first aspect, a drug administration device configured to administer a drug is provided and in one embodiment includes a housing, wherein the housing includes a first housing electrical contact and a second housing electrical contact; a dispensing mechanism configured to dispense the drug, wherein the dispensing mechanism is disposed within the housing; a drug holder configured to hold the drug; and a removable power supply configured to power the drug administration device, wherein the removable power supply includes a first power supply electrical contact and a second power supply electrical contact; and wherein (a) the housing includes a housing protection mechanism configured to be in (i) a first configuration configured to protect the first housing electrical contact when the removable power supply is not received within the housing; and (ii) a second configuration configured to enable contact between the first and second housing electrical contacts and the first and second power supply electrical contacts, respectively, when the removable power supply is received within the housing; and/or (b) the removable power supply includes a power supply protection mechanism configured to be in (i) a first configuration configured to protect the second power supply electrical contact when the removable power supply is not received within the housing; and (ii) a second configuration configured to enable contact between the first and second housing electrical contacts and the first and second power supply electrical contacts, respectively, when the removable power supply is received within the housing.

The drug administration device can have any number of variations. For example, the housing can include the housing protection mechanism, the removable power supply can include the removable power supply protection mechanism, and/or the housing protection mechanism in the first configuration can form a seal around the first housing electrical contact. In at least some embodiments, the housing protection mechanism in the first configuration can form a seal around the second housing electrical contact, and/or the housing protection mechanism can include a membrane and the first power supply electrical contact power supply can be configured to pierce the membrane such that the membrane is in the second configuration when the removable power supply is received within the housing. In at least some embodiments, the first power supply electrical contact and the second power supply electrical contact can each configured be to pierce the membrane such that the membrane is in the second configuration when the removable power supply is received within the housing.

For another example, the power supply protection mechanism in the first configuration can form a seal around the second power supply electrical contact. In at least some embodiments, the power supply protection mechanism in the first configuration can form a seal around the first power supply electrical contact, and/or the power supply protection mechanism can be a membrane and the second housing electrical contact can be configured to pierce the membrane such that the membrane is in the second configuration when the removable power supply is received within the housing. In at least some embodiments, the first housing electrical contact can be configured to pierce the membrane such that the membrane is in the second configuration when the removable power supply is received within the housing.

For yet another example, the housing protection mechanism in the first configuration can form a seal around the first housing electrical contact, and the power supply protection mechanism in the first configuration can form a seal around the second power supply electrical contact. In at least some embodiments, each of the housing protection mechanism and the power supply protection mechanism includes a membrane, and the first power supply electrical contact and the second housing electrical contact can each be configured to pierce the respective membranes such that each membrane is in the second configuration when the removable power supply is received within the housing.

For still another example, the housing protection mechanism can be configured to move from the first configuration to the second configuration by being contacted by the removable power supply when the removable power supply is inserted into the housing. In at least some embodiments, the housing protection mechanism can be configured to move along a surface of the first housing electrical contact. In at least some embodiments, the housing protection mechanism can be configured to move along a surface of the second housing electrical contact when the housing protection mechanism moves from the first configuration to the second configuration.

For another example, the power supply protection mechanism can be configured to move from the first configuration to the second configuration by being contacted by the housing when the removable power supply is inserted into the housing. In at least some embodiments, the power supply protection mechanism can be configured to move along a surface of the first power supply electrical contact. In at least some embodiments, the power supply protection mechanism can be configured to move along a surface of the second power supply electrical contact when the power supply protection mechanism moves from the first configuration to the second configuration.

For still another example, at least one of the first housing electrical contact, the second housing electrical contact, the first power supply electrical contact, and the second power supply electrical contact can be coated with a hydrophobic conductive coating.

For yet another example, the drug administration device can include at least one sensor located within the housing configured to detect an environmental parameter within the housing, and the drug administration device can include a processor configured to receive data from the at least one sensor and to modify the operation of the drug administration device in response to the data received from the at least one sensor. In at least some embodiments, the at least one sensor can include a moisture sensor, a temperature sensor, and/or a contamination sensor (e.g., a moisture sensor or a photosensor). In at least some embodiments, the processor can be configured to modify the operation of the drug administration device in response to the data received from the at least one sensor by (a) beginning a limp mode operation, wherein the limp mode operation comprises reduced functionality, when a contaminant is determined to be present; (b) indicating to a user the status of the drug administration device; (c) preventing startup of the drug administration device, when a contaminant is determined to be present; and/or (d) communicating with a remote server, when a contaminant is determined to be present. In at least some embodiments, the at least one sensor can include a moisture sensor, and the processor can be configured to modify the operation of the drug administration device when the processor receives data from the moisture sensor indicating that moisture contamination has been detected within the housing. In at least some embodiments, the at least one sensor can include a first moisture sensor located at a first position in the housing and a second moisture sensor located at a second position within the housing. The processor can be configured to receive data from the first moisture sensor and the second moisture sensor and to determine a location of moisture contamination within the housing from the data. The processor can be configured to modify the operation of the drug administration device in a different way in response to different locations of moisture contamination within the housing.

For still another example, the housing can include a conductive trace, and the at least one sensor can include a sensor that is configured to measure a conductivity of the conductive trace. In at least some embodiments, the conductive trace can be positioned across a join in the housing.

For yet another example, the housing can include an electromagnetic shield configured to prevent electromagnetic radiation interacting with the drug administration device.

For another example, the drug can include at least one of infliximab, golimumab, ustekinumab, daratumumab, guselkumab, epoetin alfa, risperidone, and paliperidone palmitate.

In another embodiment a drug administration device configured to administer a drug includes a housing; a dispensing mechanism configured to dispense the drug, wherein the dispensing mechanism is disposed within the housing; a drug holder configured to hold the drug; at least one sensor located within the housing configured to detect an environmental parameter within the housing; and a processor, wherein the processor is configured to receive data from the at least one sensor and to modify the operation of the drug administration device in response to the data received from the at least one sensor.

The drug administration device can have any number of variations. For example, the at least one sensor can include at least one contamination sensor. In at least some embodiments, the at least one contamination sensor can include a moisture sensor or a photosensor. The photosensor can be configured to detect light from the drug holder, and the processor can be configured to determine whether the drug holder contains a contaminant based on the data received from the photosensor. The drug holder can be transparent to at least a wavelength of light that the photosensor is configured to detect. The drug administration device can include a light source configured to irradiate the drug holder, and the photosensor can be configured to detect the light from the light source after it has passed through the drug holder.

For yet another example, the processor can be configured to modify the operation of the drug administration device in response to the data received from the at least one sensor by (a) beginning a limp mode operation, wherein the limp mode operation comprises reduced functionality, when a contaminant is determined to be present; (b) indicating to a user the status of the drug administration device; (c) preventing startup of the drug administration device, when a contaminant is determined to be present; and/or (d) communicating with a remote server, when a contaminant is determined to be present.

For still another example, the at least one sensor can include a moisture sensor, and the processor can be configured to modify the operation of the drug administration device when the processor receives data from the moisture sensor indicating that moisture contamination has been detected within the housing.

For another example, the at least one sensor can include a first moisture sensor located at a first position in the housing and a second moisture sensor located at a second position within the housing. In at least some embodiments, the processor can be configured to receive data from the first moisture sensor and the second moisture sensor and to determine the location of moisture contamination within the housing from the data. The processor can be configured to modify the operation of the drug administration device in a different way in response to different locations of moisture contamination within the housing.

For still another example, the housing can include a conductive trace, and the at least one sensor can include a sensor that is configured to measure the conductivity of the conductive trace. In at least some embodiments, the conductive trace can be positioned across a join in the housing.

For yet another example, the housing can include an electromagnetic shield configured to prevent electromagnetic radiation interacting with the drug administration device.

For another example, the drug can include at least one of infliximab, golimumab, ustekinumab, daratumumab, guselkumab, epoetin alfa, risperidone, and paliperidone palmitate.

In another embodiment, a drug administration device configured to administer a drug includes a housing; a dispensing mechanism configured to dispense the drug, wherein the dispensing mechanism is disposed within the housing; a removable power supply configured to power the drug administration device, wherein the housing is configured to receive the removable power supply; a first sensor, wherein the first sensor is configured to measure a parameter indicative of the remaining charge of the removable power supply and output data; and a processor, wherein the processor is configured to receive the data from the first sensor and modify the operation of the drug administration device in response to the data received from the first sensor.

The drug administration device can vary in any number of ways. For example, the housing can be configured to receive a second power supply configured to power the drug administration device. In at least some embodiments, the drug administration device can include a second sensor configured to determine the remaining charge of the second power supply, and the processor can be configured to receive data from the second sensor and modify the operation of the drug administration device in response to the data received from the second sensor. In at least some embodiments, the second power supply can be a primary cell. In at least some embodiments, the drug administration device can include the second power supply, and the second power supply can be disposed within the housing. The second power supply can be configured to be wirelessly charged. The drug administration device can be configured to charge the second power supply using the removable power supply when the removable power supply is received within the housing. The second power supply can be configured to power the drug administration device for a predetermined period of time, and the removable power supply can be configured to be chargeable from no charge to full charge within the predetermined period of time.

For another example, modifying the operation of the drug administration device can include the processor being configured to initiate an alert when the remaining charge of the removable power supply reaches a first threshold level. For yet another example, the processor can be configured to initiate an alert when the remaining charge of the second power supply reaches a second threshold level. For still another example, the removable power supply can include removable power supply identity data, the processor can be configured to receive the removable power supply identity data when the removable power supply is received within the housing, and the processor can be configured to modify the operation of the drug administration device in response to the removable power supply identity data. For another example, the removable power supply can include a memory configured to receive data. For still another example, the dispensing mechanism can be configured to be powered by the removable power supply.

For yet another example, the dispensing mechanism can include a device operation prevention mechanism. In at least some embodiments, the removable power supply can include removable power supply identity data, the processor can be configured to receive the removable power supply identity data when the removable power supply is received within the housing, the processor can be configured to modify the operation of the drug administration device in response to the removable power supply identity data, and modifying the operation of the drug administration device can include enabling the device operation prevention mechanism to prevent the dispensing mechanism from being powered by the removable power supply when the removable power supply identity data indicates that the removable power supply is not compatible with the drug administration device. The device operation prevention mechanism can be configured to prevent the dispensing mechanism from being powered by the removable power supply when the remaining charge in the removable power supply falls below a third threshold level. The third threshold level can be enough charge for the dispensing mechanism to complete a full drug administration sequence.

For still another example, the drug dispensing mechanism can be configured to be operated manually. In at least some embodiments, the dispensing mechanism comprises a device operation prevention mechanism, and the device operation prevention mechanism can be configured to prevent the dispensing mechanism from being operated manually when the remaining charge of the removable power supply is above a fourth threshold level.

For another example, the drug can include at least one of infliximab, golimumab, ustekinumab, daratumumab, guselkumab, epoetin alfa, risperidone, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an autoinjector;

FIG. 2 is a schematic view of a second type of drug administration device, namely an infusion pump;

FIG. 3 is a schematic view of a third type of drug administration device, namely an inhaler;

FIG. 4 is a schematic view of a general drug administration device;

FIG. 5 is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate;

FIG. 9 is a schematic view of one embodiment of a drug administration device with multiple sensors located within the housing;

FIG. 10a is a schematic view of one embodiment of a housing protection mechanism in a first configuration;

FIG. 10b is a schematic view of the embodiment of FIG. 10a with the housing protection mechanism in a second configuration;

FIG. 11 is a schematic view of one embodiment of a housing protection mechanism in a first configuration;

FIG. 12 is a schematic view of one embodiment of a drug administration device comprising a photosensor and a light source configured to detect contamination within the drug housing; and

FIG. 13 is a schematic view of another embodiment of a drug administration device including a dispensing mechanism that is both electronically and manually operable.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

In relation to how a drug is administered to the patient, there are various dosage forms that can be used. For example, these dosage forms may include parenteral, inhalational, oral, ophthalmic, topical and suppository forms of one or more drugs.

The dosage forms can be administered directly to the patient via a drug administration device. There are a number of different types of drug administration devices commonly available for delivery of the various dosage forms including: syringes, injection devices (e.g. autoinjectors, jet injectors, and infusion pumps), and inhalers.

It can be desirable to monitor compliance with the guidance that is associated with the drugs that are administered to a patient in various dosage forms. This can provide assurance that correct procedures are being followed and avoid the adoption of incorrect and potentially dangerous approaches. Further, this can also enable optimisation of the administration of the drug to the patient.

Any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), and Invega Trinza® (paliperidone palmitate).

Examples of various types of drug administration devices, namely: an autoinjector 100, an infusion pump 200 and an inhaler 300, are described below with reference to the hereinbefore referenced figures.

### Autoinjector

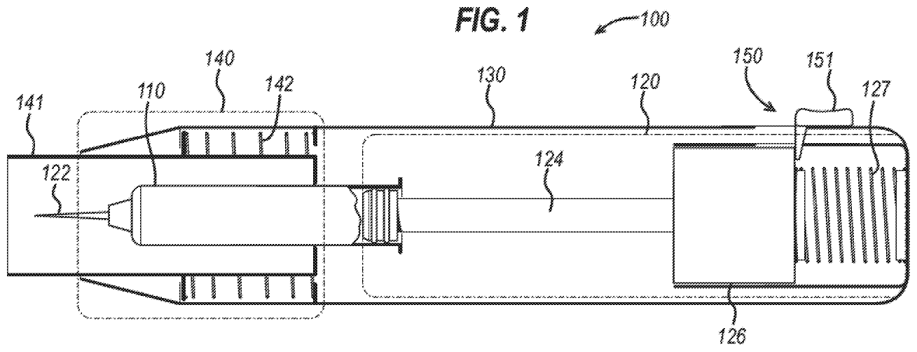

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurised gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

Whilst the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Autoinjectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient.

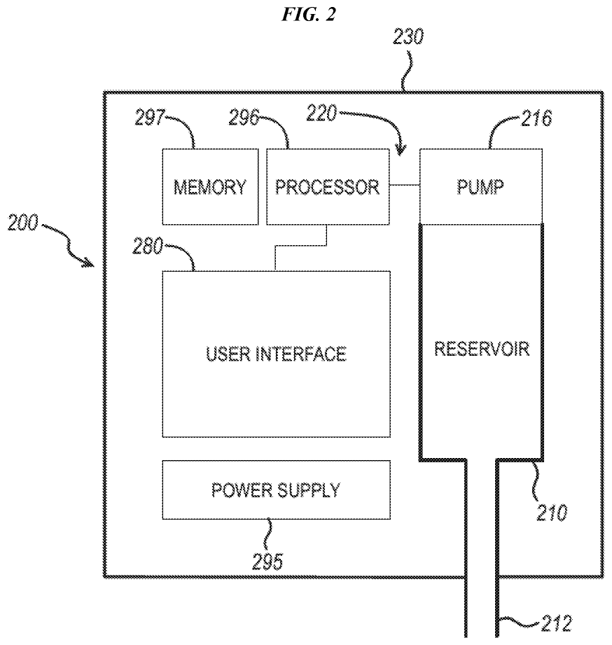

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g. patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre-programmed limits. The programmed parameters for controlling the pump 216 are stored in and retrieved from the memory 297 which is in communication with the processor 296. The user interface 280 may take the form of a touch screen or a keypad.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g. for use at a patient's bedside, or it may be an ambulatory infusion pump which is designed to be portable or wearable. An integral power supply 295 is particularly beneficial for ambulatory infusion pumps.

While the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

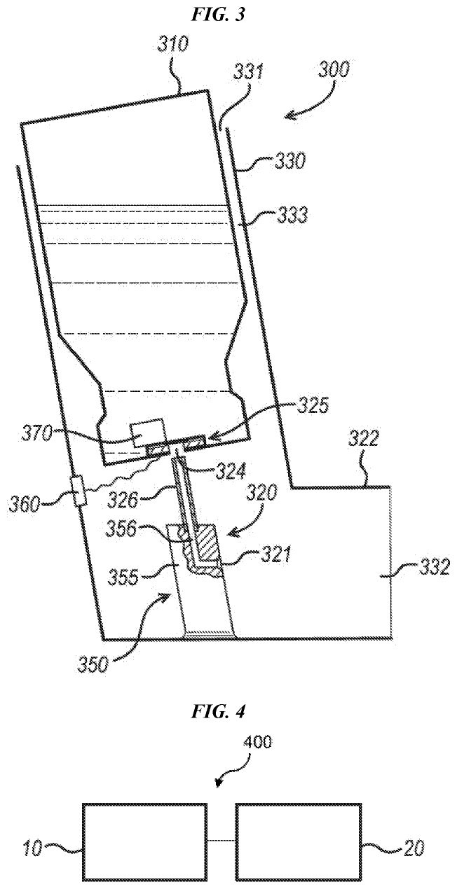

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurised gas for pressurising the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurised gas propels the drug in solution or suspension with the suitable liquid out of the drug holder 310 through the opening 324 at high speed. The high speed passage of the liquid through the narrow opening 324 causes the liquid to be atomised, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomisation of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomised by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

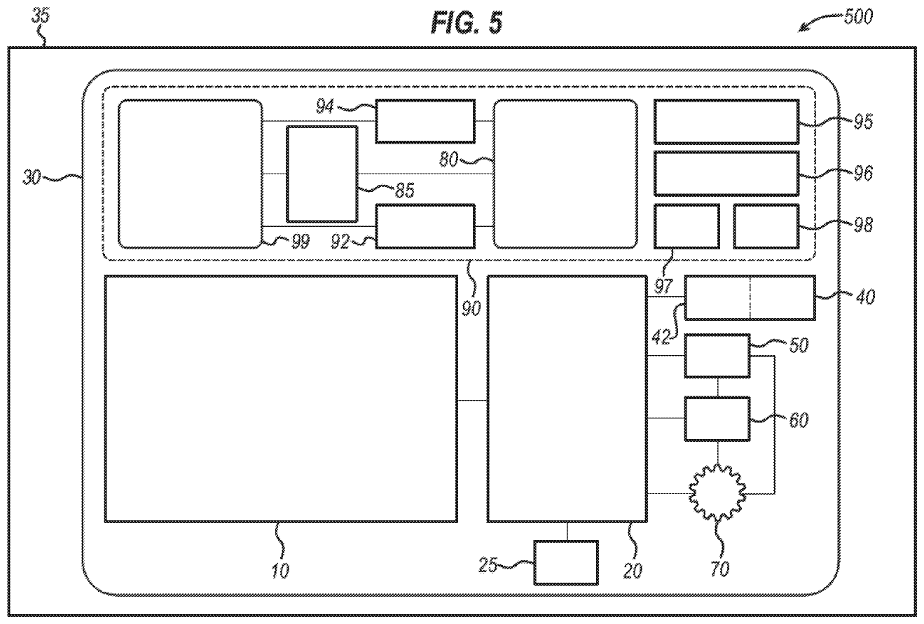

FIG. 4 is a generalised schematic view of such a universal drug administration device 400, and FIG. 5 is an exemplary embodiment of such a universal drug administration device 500.

As shown in FIG. 4, drug administration device 400 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5 shows a further universal drug administration device 500 which includes a number of additional features. A person skilled in the art understands that these additional features are optional for different embodiments, and can be utilised in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 4, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 50. The dose volume can be set to one specific value of a plurality of predefined discrete dose volumes, or any value of predefined dose volume within a range of dose volumes.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20. Similarly, an environment sensor 94 is provided which is configured to sense information relating to the environment in which the device 500 is present, such as the temperature of the environment, the humidity of the environment, location, and time. There may be a dedicated location sensor 98 which is configured to determine the geographical location of the device 500, e.g. via satellite position determination, such as GPS. The device 500 also includes a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilise a liquid dosage form. It will be appreciated, however that other dosage forms are available.

One such common dosage form is a tablet. The tablet may be formed from a combination of the drug and an excipient that are compressed together. Other dosage forms are pastes, creams, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilised with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), and Invega Trinza® (paliperidone palmitate).

### Drug Housing

As described above, a dosage form can be provided in a holder that is appropriate for the particular dosage form being utilised. For example, a drug in a liquid dosage form can be held prior to administration within a holder in the form of a vial with a stopper, or a syringe with a plunger. A drug in solid or powder dosage form, e.g. as tablets, may be contained in a housing which is arranged to hold the tablets securely prior to administration.

The housing may comprise one or a plurality of drug holders, where each holder contains a dosage form, e.g. the drug can be in a tablet dosage form and the housing can be in the form of a blister pack, where a tablet is held within each of a plurality of holders. The holders being in the form of recesses in the blister pack.

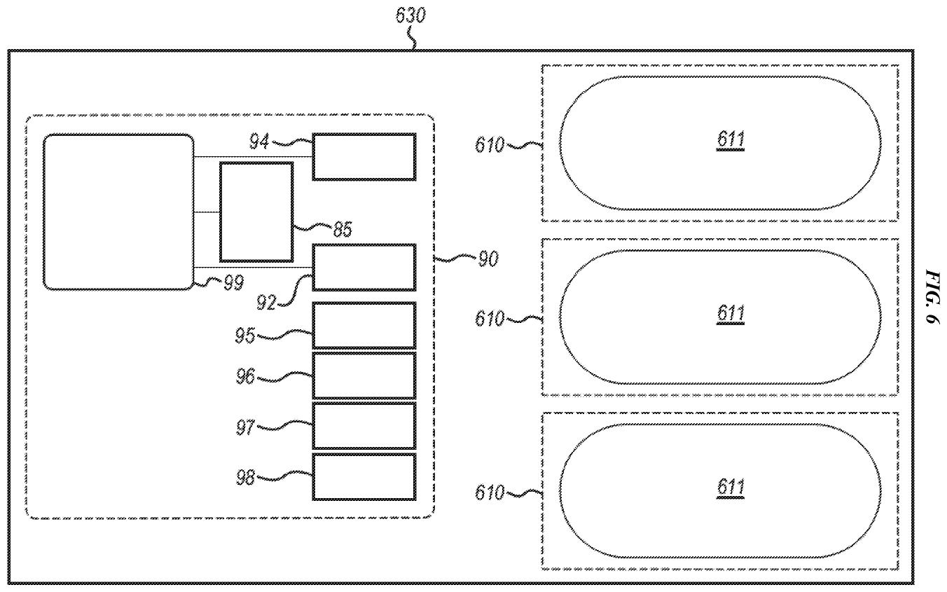

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g. via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 85, as described above.

### Electronic Communication

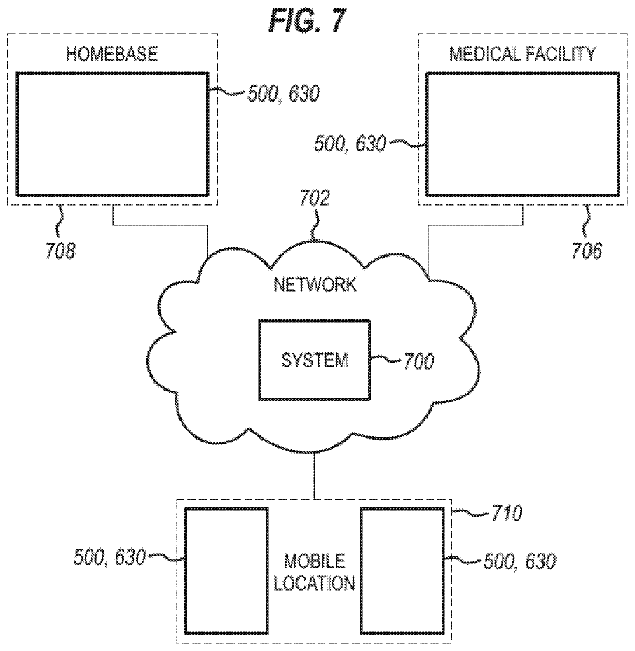

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g. a hospital or other medical care center, a home base 708 (e.g. a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g. through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g. the identity of the user and/or the location from which the user is accessing the system. To that end, each user can have a unique username, password, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g. the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g. as a touch screen) or indirectly (e.g. via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

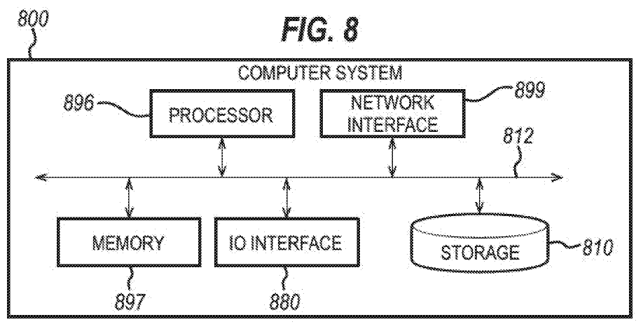

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g. static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (TO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The TO interface(s) 880 include one or more interface components to connect the computer system 800 with other electronic equipment. For example, the TO interface(s) 880 can include high speed data ports, such as universal serial bus (USB) ports, 1394 ports, Wi-Fi, Bluetooth, etc. Additionally, the computer system can be accessible to a human user, and thus the TO interface(s) 880 can include displays, speakers, keyboards, pointing devices, and/or various other video, audio, or alphanumeric interfaces. The storage device(s) 810 include any conventional medium for storing data in a non-volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g. a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 799 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g. contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For example, the memory 897 and storage device 810 can be integrated together or the communications interface 899 can be omitted if communication with another computer system is not necessary.

### A. Implementations

Embodiments of drug administration devices described herein can be configured to be used in environments where the drug administration device may be exposed to moisture or other contaminants, which may reduce the risk of patient harm resulting from incorrect application of the drug.

Embodiments of drug administration devices described herein can be configured to not leave a user unable to administer a drug from the drug administration device when the drug is needed, which may improve patient compliance and therefore safety.

In an exemplary embodiment, a drug administration device configured to administer a drug includes a housing including a first housing electrical contact and a second housing electrical contact, a dispensing mechanism contained within the housing, a drug holder configured to hold the drug, and a removable power supply configured to power the drug administration device and including a first power supply electrical contact and a second power supply electrical contact. The housing includes a housing protection mechanism and/or the power supply can include a power supply protection mechanism. The housing protection mechanism is configured to be in a first configuration that protects the first housing electrical contact when the removable power supply is not received within the housing, and in a second configuration that enables contact between the first and second housing electrical contacts and the first and second power supply electrical contacts, respectively, when the removable power supply is received within the housing. The power supply protection mechanism is configured to be in a first configuration that protects the second power supply electrical contact when the removable power supply is not received within the housing and in a second configuration that enables contact between the first and second housing electrical contacts and the first and second power supply electrical contacts, respectively, when the removable power supply is received within the housing.

By protecting the electrical contacts on the housing and/or the removable power supply, the drug administration device can be used, and the power supply can be replaced in environments where there is a risk of either the housing or the removable power supply coming into contact with contaminants. This may be particularly suitable to wearable drug administration devices where the drug administration device may necessarily be taken and used wherever the patient goes, and this may be particularly suitable to drug administration devices that consume a large amount of power and so require regular removal and/or replacement of the removable power supply.

The form of the drug administration device can be any of the drug administration devices described herein.

The housing of the drug administration device is the external portion of the drug administration device. The housing of the drug administration device can be configured to keep contaminants out of an internal portion of the drug administration device, where the internal portion of the drug administration device includes all parts of the drug administration device disposed within the housing. In particular, the housing can be configured to be water resistant, e.g., waterproof to IPX2, IPX3, IPX4, IPX5, IPX6, IPX7, IPX8 or more, according to the IEC standard 60529. The housing can combine with a portion of the removable power supply when the removable power supply is received within the housing in order to provide an external portion of the drug administration device and, if the housing is configured to be water resistant or waterproof, the required water resistance or water proofing.

The drug holder of the drug administration device can be of any suitable form for holding the drug. As noted herein, such suitable forms include a vial or a syringe.

The removable power supply of the drug administration device can be a primary battery that is configured to be used once. Alternatively, the removable power supply can be a secondary battery that is configured to be recharged. The primary battery can include, e.g., a zinc-carbon battery or an alkaline battery. The secondary battery can include, e.g., a nickel-cadmium cell, a nickel-zinc cell, a nickel metal hydride cell, or a lithium-ion cell.

The first and second housing electrical contacts and the first and second power supply electrical contacts of the drug administration device are configured such that electrical contact can be formed between the first housing electrical contact and the first power supply electrical contact, and the second housing electrical contact and the second power supply electrical contact. When the removable power supply is received within the housing, the first housing electrical contact and the first power supply electrical contact, and the second housing electrical contact and the second power supply electrical contact are in contact with each other such that an electrical circuit between the housing and the removable power supply is completed. This may result in the provision of power from the removable power supply to other components of the drug administration device.

The protection mechanism(s) are configured to be in a first configuration when the removable power supply is not received in the housing. The first configuration is the configuration in which the protection mechanism provides its protection function for the respective electrical contact. The first configuration may be configured to reduce or prevent liquid ingress to the respective electrical contact. Alternatively, or in addition, the first configuration may be configured to reduce or prevent solid particle ingress to the respective electrical contact. The reduction in liquid and/or solid particle ingress to the respective electrical contact is a reduction relative to the ingress that would occur in the absence of the protection mechanism. In this way the protection mechanism(s) help to protect the electrical contacts from contamination.

The protection mechanism(s) are configured to be in a second configuration when the removable power supply is received within the housing. The second configuration is the configuration in which the protection mechanism enables connection between the respective electrical contacts. This can be achieved by the protection mechanism being moved from the first configuration to the second configuration by the action of combining the removable power supply with the housing so that the removable power supply is received within the housing. The protection mechanism can be configured to be manually moveable between the first configuration and the second configuration in order to enable the connection.

The housing protection mechanism is configured to move between the first and second configuration by being contacted by the removable power supply when the removable power supply is inserted into the housing. The housing protection mechanism is configured to move along a surface of the first housing electrical contact, and possibly move along a surface of the second housing electrical contact, when the housing protection mechanism moves from the first configuration to the second configuration. By moving along the surface(s) of the electrical contact(s), the housing protection mechanism physically scrapes or wipes off contaminants that may have gathered on the surface(s) of the electrical contact(s), thereby providing a cleaner surface of the electrical contact when the removable power supply is received within the housing, which may ensure that there is an efficient electrical connection between the first housing electrical contact and the first power supply electrical contact, and, if the housing protection mechanism also moves along the surface of the second housing electrical contact, between the second housing electrical contact and the second power supply electrical contact.

The housing protection mechanism can be configured to form a seal around the first housing electrical contact. Herein, a seal refers to a continuous contact between two surfaces that prevents a contaminant passing between the two surfaces at the point of contact. The point of contact can be a vulnerable area for ingress of contaminants. The contaminant can be a liquid and/or a solid. The seal is configured to prevent moisture passing between the two surfaces at the point of contact. The seal can be formed between the housing protection mechanism and the housing itself. By forming a seal around the housing electrical contacts, the housing protection mechanism can prevent moisture from contacting the first and second housing electrical contacts and potentially creating a circuit between the first and second housing electrical contacts when the removable power supply is not received within the housing, which may reduce the risk of the drug administration device malfunctioning when used in environments in which there is a risk that the drug administration device will come into contact with a contaminant, for example moisture.

Further, the housing protection mechanism can be configured to seal the first housing electrical contact, e.g., sealing the first electrical contact to prevent contaminant ingress from a range of directions. In this regard, the housing can protect the first housing electrical contact from contaminant ingress in certain directions, and the sealing of the first electrical contact protects the first housing electrical contact from contaminant ingress in the remaining directions, e.g., by forming a seal around the first housing electrical contact so as to prevent ingress of a contaminant at this interface.

The seal can be formed by covering the first housing electrical contact with a sealing layer. The sealing layer can include an elastomeric material. The use of an elastomeric material may assist in the housing protection mechanism being moveable from the first configuration to the second configuration.

The housing protection mechanism can be in the form of a membrane. The housing protection mechanism can include a membrane in the form of a layer that extends over the first housing electrical contact. The membrane can result in the first housing electrical contact being sealed so as to protect the first housing electrical contact from contaminant ingress.

Herein, membrane refers to a pliable sheet or film of material. The membrane can include one or more layers. The membrane is substantially impermeable to the contaminant of concern, e.g., the membrane can be impermeable to water. The membrane of the drug administration device can be a synthetic membrane. The membrane of the drug administration device can include an elastomeric material. The membrane of the drug administration device can include a polymer, for example cellulose acetate, nitrocellulose, cellulose esters, polysulfone, polyehtersulfone, polyacrilonitrile, polyamide, polyimide, polyethylene, polypropylene, polytetrafluoroethylene, polyvinylidene fluoride, polyvinylchloride, etc.

The first power supply electrical contact can be configured to pierce the housing protection mechanism. In this embodiment, the housing protection mechanism is configured to be pierceable. The first power supply electrical contact being configured to pierce the housing protection mechanism is particularly relevant when the housing protection mechanism is in the form of a membrane. The action of piercing the housing protection mechanism forces the housing protection mechanism into the second configuration, such that contact can be made between the electrical contacts. When multiple housing electrical contacts are protected by a pierceable housing protection mechanism, multiple respective power supply electrical contacts can be configured to pierce the relevant housing protection mechanism. The use of a piercing action also has the benefit of the housing protection mechanism moving along a surface of the electrical contact and so removing contaminants before a connection is made.

The respective power supply electrical contact can be configured to pierce the housing protection mechanism by including at least one sharp or pointed feature. By protecting the housing electrical contacts with a pierceable housing protection mechanism and having the power supply electrical contacts configured to pierce the housing protection mechanism, the drug administration device can be configured to reduce the possibility that there are contaminants on the respective housing electrical contact that could inhibit the electrical connection between the housing electrical contacts and the power supply electrical contacts.

The housing protection mechanism can be configured to protect just one electrical contact, e.g., the first housing electrical contact as detailed above. In this case, there can be an additional housing protection mechanism to protect the other electrical contact. Where there are additional electrical contacts, these can also be protected by additional housing protection mechanisms. In this manner, each of the contacts can be protected separately. If a problem of contaminant ingress is associated with one of these contacts then it will not affect the other contacts. Further, the isolation of each of the contacts will ensure that the contaminant ingress does not create an undesired short circuit between the contacts. When there are a plurality of housing protection mechanisms, each of the housing protection mechanisms can be of the same form. Alternatively, the housing protection mechanisms can be of different forms. The features described in relation to the housing protection mechanism protecting the first housing electrical contact apply equally to each of the additional housing protection mechanisms.

Alternatively to a single housing protection mechanism being configured to protect a single electrical contact, a single housing protection mechanism can be configured to protect multiple contacts, which may efficiently provide protection from contaminant ingress.

The first housing electrical contact can be coated with a hydrophobic coating. Herein, “hydrophobic” means that the contact angle of a water droplet on the surface of the coating exceeds 90°, 100°, 110°, or 120°, as measured by the static sessile drop method. The hydrophobic coating is configured so as to not substantially affect a conductivity across the coating layer. This can be achieved by using a conductive hydrophobic coating. Herein, “conductive” means that the electrical resistivity of the coating is less than 10⁻⁴ Ωm, 10⁻⁵ Ωm, 10⁻⁶ Ωm, 10⁻⁷ Ωm, or 10⁻⁸ Ωm at 20° C. The hydrophobic conductive coating can include, e.g., carbon nano-tube structures, silica nano-coating, polycrystalline gold, polycrystalline silver, or polycrystalline copper. The hydrophobic conductive coating can be achieved by etching a surface of the electrical contact. By coating the first housing electrical contact with a hydrophobic coating, the amount of moisture contamination that gathers on a surface of the first housing electrical contact is reduced. By coating the first housing electrical contact with a hydrophobic coating in combination with configuring the housing protection mechanism to move along a surface of the first housing electrical contact when moving between the first configuration and the second configuration, the surface of the first housing electrical contact is more effectively cleaned when the removable power supply is inserted into the housing.

The features described in relation to the housing protection mechanism equally apply to the power supply protection mechanism, when it is present in addition to, or alternatively to, the housing protection mechanism. For example, the first housing electrical contact can be protected by a housing protection mechanism, and the second power supply electrical contact can be protected by a power supply protection mechanism. In addition, both of these protection mechanisms may be configured to be pierceable. Accordingly, the second housing electrical contact and the first power supply electrical contact can be configured to pierce the power supply protection mechanism and the housing protection mechanism, respectively, to form the electrical contacts, which may provide a form of protection for the housing electrical contacts and the power supply electrical contacts.

The power supply protection mechanism is configured to move between the first and second configurations by being contacted by the housing when the removable power supply is inserted into the housing. The power supply protection mechanism can be configured to move along a surface of the first power supply electrical contact and, possibly move along a surface of the second power supply electrical contact, when the power supply protection mechanism moves from the first configuration to the second configuration. By moving along the surface(s) of the electrical contact(s) the power supply protection mechanism physically scrapes or wipes off any contaminants that may have gathered on the surface(s) of the electrical contact(s), thereby providing a cleaner surface of the electrical contact when the removable power supply is received within the housing, which may ensure that there is an efficient electrical connection between the first power supply electrical contact and the first housing electrical contact, and, if the power supply protection mechanism also moves along the surface of the second power supply electrical contact, between the second power supply electrical contact and the second housing electrical contact.

The power supply protection mechanism can be configured to form a seal around the first power supply electrical contact. The seal can be formed between the power supply protection mechanism and the removable power supply itself. The power supply protection mechanism can be configured to form a seal around the first power supply electrical contact and the second power supply electrical contact. By forming a seal around the power supply electrical contacts, the power supply protection mechanism can prevent moisture from contacting the first and second power supply electrical contacts and potentially creating a circuit between the first and second power supply electrical contacts when the removable power supply is not received within the housing, which may reduce the risk of the drug administration device malfunctioning when used in environments in which there is a risk that the drug administration device will come into contact with a contaminant, for example moisture.

Further, the power supply protection mechanism can be configured to seal the first power supply electrical contact, e.g., sealing the first electrical contact to prevent contaminant ingress from a range of directions. In this regard, the removable power supply can protect the first power supply electrical contact from contaminant ingress in certain directions, and the sealing of the first electrical contact protects the first power supply electrical contact from contaminant ingress in the remaining directions, e.g., by forming a seal around the first power supply electrical contact so as to prevent ingress of a contaminant at this interface.

The seal can be formed by covering the first power supply electrical contact with a sealing layer. The sealing layer can include an elastomeric material. The use of an elastomeric material may assist in the power supply protection mechanism being moveable from the first configuration to the second configuration.

The power supply protection mechanism can be in the form of a membrane. The power supply protection mechanism can include a membrane in the form of a layer that extends over the first power supply electrical contact. The membrane can result in the first power supply electrical contact being sealed so as to protect the first power supply electrical contact from contaminant ingress.

The first housing electrical contact can be configured to pierce the power supply protection mechanism. In this embodiment, the power supply protection mechanism is configured to be pierceable. The first housing electrical contact being configured to pierce the power supply protection mechanism is particularly relevant when the power supply protection mechanism is in the form of a membrane. The action of piercing the power supply protection mechanism forces the power supply protection mechanism into the second configuration, such that contact can be made between the electrical contacts. When multiple power supply electrical contacts are protected by a pierceable power supply protection mechanism, multiple respective housing electrical contacts can be configured to pierce the relevant power supply protection mechanism. The use of a piercing action also has the benefit of the power supply protection mechanism moving along a surface of the electrical contact and so removing contaminants before a connection is made.

The respective housing electrical contact can be configured to pierce the power supply protection mechanism by comprising at least one sharp or pointed feature. By protecting the power supply electrical contacts with a pierceable power supply protection mechanism and having the housing electrical contacts configured to pierce the power supply protection mechanism, the drug administration device can be configured reduce the possibility that there are contaminants on the respective power supply electrical contact that could inhibit the electrical connection between the power supply electrical contacts and the housing electrical contacts.

The power supply protection mechanism can be configured to protect just one electrical contact, e.g., the first power supply electrical contact as detailed above. In this case, there can be an additional power supply protection mechanism to protect the other electrical contact. Where there are additional electrical contacts, these can also be protected by additional power supply protection mechanisms. In this manner, each of the contacts can be protected separately. If a problem of contaminant ingress is associated with one of these contacts then it will not affect the other contacts. Further, the isolation of each of the contacts will ensure that the contaminant ingress does not create an undesired short circuit between the contacts. When there are a plurality of power supply protection mechanisms, each of the power supply protection mechanisms can be of the same form. Alternatively, the power supply protection mechanisms can be of different forms. The features described in relation to the power supply protection mechanism protecting the first power supply electrical contact apply equally to each of the additional power supply protection mechanisms.

Alternatively to a single power supply protection mechanism being configured to protect a single electrical contact, a single power supply protection mechanism can be configured to protect multiple contacts, which may efficiently provide protection from contaminant ingress.

The first power supply electrical contact can be coated with a hydrophobic conductive coating. By coating the first power supply electrical contact with a hydrophobic conductive coating, the amount of moisture contamination that gathers on a surface of the first power supply electrical contact is reduced. By coating the first power supply electrical contact with a hydrophobic conductive coating in combination with configuring the power supply protection mechanism to move along a surface of the first power supply electrical contact when moving between the first configuration and the second configuration, the surface of the first power supply electrical contact is more effectively cleaned when the removable power supply is inserted into the housing.

The housing protection mechanism can, in the first configuration, be configured to form a seal around the first housing electrical contract and the power supply protection mechanism can, in the first configuration, be configured to form a seal around the second power supply electrical contact. By forming a seal around the first housing electrical contact and the second power supply electrical contact, the protection mechanisms can prevent moisture from contacting the first housing electrical contact and the second power supply electrical contact and potentially creating a circuit between the first and second power supply electrical contacts or the first and second housing electrical contacts when the removable power supply is not received within the housing, which may reduce the risk of the drug administration device malfunctioning when used in environments in which there is a risk that the drug administration device will come into contact with a contaminant, for example moisture.

The power supply protection mechanism and the housing protection mechanism can each be a membrane, and the second housing electrical contact and the first power supply electrical contact can each be configured to pierce the respective membranes such that each membrane is in the second configuration when the power supply is received within the housing. The second housing electrical contact and the first power supply electrical contact can be configured to pierce the respective membrane by including at least one sharp or pointed feature. By protecting the first housing electrical contact and the second power supply electrical contact with the membrane, the drug administration device may ensure that there are no contaminants on surfaces of the electrical contacts that would result in a poorer electrical connection between the power supply electrical contacts and the housing electrical contacts and may ensure that there is no way for a contaminant to form a circuit between the first and second power supply electrical contacts or the first and second housing electrical contacts.

The second housing electrical contact can be coated with a hydrophobic conductive coating. By coating the second housing electrical contact with a hydrophobic conductive coating, the amount of moisture contamination that gathers on a surface of the second housing electrical contact is reduced. By coating the second housing electrical contact with a hydrophobic conductive coating in combination with configuring the housing protection mechanism to move along a surface of the second housing electrical contact when moving between the first configuration and the second configuration, the surface of the second housing electrical contact is more effectively cleaned when the removable power supply is inserted into the housing.

The second power supply electrical contact can be coated with a hydrophobic conductive coating. By coating the second power supply electrical contact with a hydrophobic conductive coating, the amount of water contamination that gathers on the surface of the second power supply electrical contact is reduced. By coating the second power supply electrical contact with a hydrophobic conductive coating in combination with configuring the power supply protection mechanism to move along the surface of the second power supply electrical contact when moving between the first configuration and the second configuration, the surface of the second power supply electrical contact is more effectively cleaned when the removable power supply is inserted into the housing.

In another exemplary embodiment, a drug administration device configured to administer a drug includes a housing, a dispensing mechanism configured to dispense the drug and being disposed within the housing, a drug holder configured to hold the drug, at least one sensor located within the housing and being configured to detect an environmental parameter within the housing, and a processor configured to receive data from the at least one sensor and to modify the operation of the drug administration device in response to the data received from the at least one sensor.

The drug administration device can be any form of drug administration devices described herein.

The at least one sensor configured to detect an environmental parameter can include, e.g., a hygrometer, a temperature sensor, a location sensor, a time sensor, etc. The temperature sensor can be, e.g., a thermistor, a thermocouple, a resistance thermometer, or a silicon bandgap temperature sensor. The location sensor can be configured to determine a geographical location of the drug administration device by satellite position determination, such as GPS. The time sensor can be, e.g., an electric clock.

The drug administration device is configured to adapt to its internal environment, which may allow the drug administration device to avoid actions that could, as a result of the internal environment of the drug administration device, compromise the safety of a user of the drug administration device or of the drug administration device.

The at least one sensor can be configured to directly or indirectly detect a contaminant. Such a sensor can be termed a contamination sensor. A contaminant is anything the presence of which should be avoided. As noted herein, a contaminant may be a liquid or a solid (in particular in the form of solid particles). The sensor can be a moisture sensor, a pressure sensor, a photosensor, a current sensor, a voltage sensor, and/or a resistance sensor. The moisture sensor can be, e.g., a hygrometer or a water sensor. The pressure sensor can be, e.g., a piezoresistive strain gauge pressure sensor, a capacitive pressure sensor, an electromagnetic pressure sensor, a piezoelectric pressure sensor, a strain-gauge pressure sensor, an optical pressure sensor, a potentiometric pressure sensor, a force balancing pressure sensor, a resonant pressure sensor, a thermal pressure sensor, or an ionization pressure sensor. An increase in pressure determined by the pressure sensor can indicate the presence of a contaminant in the drug administration device.

The at least one sensor can be used in conjunction with a processor to determine if a contaminant is present. The processor can be configured to compare data produced by the sensor to a threshold band. If the data produced by the sensor falls outside of the threshold band then it can be concluded by the processor that there may be a contaminant present in the drug administration device. The threshold band can be preselected based on the data output of the sensor when there is no contaminant present in the drug administration device.

The at least one contamination sensor can include a photosensor configured to detect light from the drug administration device's drug holder. The processor can be configured to determine whether the drug holder contains a contaminant based on the data from the photosensor. By providing the processor with information regarding light from the drug holder, the processor can be configured to identify forms of contamination within the drug holder. The contamination can be a physical contamination in the form of, e.g., solid particles, bubbles of gas, or liquid droplets. The contamination can be a chemical contamination that changes a colour or opacity of the drug within the drug holder. The contamination can be a result of the aging of the drug. The processor can be configured to determine whether the drug holder contains a contaminant based on the data from the photosensor by comparing the data received from the photosensor to a standard value. The standard value for the specific drug and drug holder present in the drug administration device can be stored in a memory of the drug administration device. The processor can be configured to determine a possible contamination by measuring a deviation in the light detected by the photosensor, either compared to the standard value or by a deviation over time, or when the drug holder and/or drug is replaced or installed. A significant deviation from the standard value means that there is a contaminant present in the drug holder. Herein, a significant deviation may be a change in intensity of a given wavelength of 50%, 40%, 30%, 20%, 10%, 5%, 4%, 3%, 2%, 1%, or less, as selected as appropriate for a particular drug holder and drug therein. By being able to identify contamination in the drug holder, the drug administration device may prevent a user of the drug administration device from administering the drug when the drug holder contains a contaminant and thereby reduce a risk of harm caused by contamination in the drug holder. By being able to identify contamination in the drug holder, the drug administration device, e.g., the processor thereof, can be configured initiate an alert to the user of the drug administration device or to a medical professional to trigger performance of a corrective action such as replacing the drug holder with a new drug holder, replacing the drug administration device with a new drug administration device, etc.

The drug holder can be transparent to at least a wavelength of light that the photosensor is configured to detect, e.g., the drug holder may attenuate light at the wavelength of interest by less than 10%, less than 5%, less than 2%, or less than 1%. The photosensor can be configured to detect a wavelength in the infra-red or visible part of the light spectrum. By configuring the drug holder to be transparent to the wavelength of the light that the photosensor is configured to detect, the photosensor can be positioned outside of the drug holder, which may reduce the complexity and/or cost of the drug holder. A lot of drugs are liquid, therefore positioning the photosensor outside of the drug holder means that a non-waterproof photosensor can be used.

The drug administration device can include a light source configured to irradiate the drug holder, and the photosensor can be configured to detect the light from the light source after the light has passed through the drug holder. Herein, the light is considered to have passed through the drug holder when the light has passed through any portion of the drug holder, including where the light has entered the drug holder and is then reflected by a body within the drug holder causing the light to exit the drug holder in a direction substantially opposite to the direction the light entered. The light source is configured to irradiate light at a wavelength that the photosensor is configured to detect. The photosensor can be positioned on one side of the drug holder facing the drug holder with the light source positioned on the other side of the drug holder and directed at the photosensor through at least a portion of the drug holder. In such an embodiment, the processor can be configured determine a contamination by a deviation in the light being detected by the photosensor. The photosensor can be positioned adjacent to the light source with both the photosensor and the light source orientated in the same direction.

The wavelength detected by the photosensor, and emitted by the light sensor, is chosen to correspond to a wavelength that is affected by the presence of the contaminant of interest.

The processor can be configured to modify operation of the drug administration device in response to data received from the at least one sensor by beginning a limp mode operation when a contaminant is determined by the processor to be present. Herein, limp mode operation refers to the drug administration having a reduced functionality, such as the drug administration device not being able to administer the drug or being able to only administer a reduced dose of the drug. This reduced functionality may enable the drug administration device to avoid actions that may compromise the safety of the patient due to the presence of a contaminant, thereby improving patient safety.

The exact form of the limp mode can be determined by the processor comparing a degree or form of contamination with a predefined response. The predefined response can be stored in a memory of the drug administration device.

The processor can be configured to modify the operation of the drug administration device in response to the data received from the at least one sensor by indicating to a user of the drug administration device a status of the drug administration device. The processor can be configured to indicate to the user the status of the drug administration device via a device indicator or user interface of the drug administration device. Additionally or alternatively, the processor can be configured to indicate to the user of the drug administration device the status of the drug administration device by initiating a wireless communication, e.g., via a communications interface thereof, to a wireless device owned by or otherwise associated with the user, for instance a smartphone. This wireless communication may enable the drug administration device to warn the drug administration device's user of the presence of a contaminant and therefore enable the user of the drug administration device to take a required action. The required action may be removing the contaminant or contacting a medical professional for assistance.

The processor can be configured to modify the operation of the drug administration device in response to the data received from the at least one sensor by preventing startup of the drug administration device when a contaminant is determined to be present. In other words, all operations of the drug administration device can be prevented. A contaminant may result in the drug administration device not working as intended, so the prevention of the operation of the drug administration device results in improved user safety.

The processor can be configured to modify the operation of the drug administration device in response to the data received from the at least one sensor by communicating with a remote server, when a contaminant is determined to be present. The processor can be configured to communicate with the remote server via a communications interface of the drug administration device. The remote server can, in response to the communication from the drug administration device, be configured to initiate the ordering of a replacement drug holder, thereby reducing the amount of time that the drug administration device's user does not have access to the contaminant-free drug. The remote server can, in response to the communication from the drug administration device, be configured to alert a medical professional to the status of the drug administration device to enable the medical professional to take any desired corrective action, such as contacting the user, initiate the ordering of a replacement drug holder, etc.

The at least one sensor can include a moisture sensor, and the processor can be configured to modify the operation of the drug administration device when the processor receives data from the moisture sensor indicating that a critical level of moisture has been detected within the housing. Moisture can cause the electric components of the drug administration device to malfunction. A malfunctioning drug administration device is a risk to user safety. By determining the presence of moisture within the housing, the drug administration device may improve user safety.

The at least one sensor can include a first moisture sensor in a first position in the housing and a second moisture sensor located in a second position in the housing. A drug administration device will typically have multiple internal portions within its housing, and each internal portion can be sealed from the other internal portions. As different internal portions contain different components, the impact of moisture contamination within each internal portion can vary. By having multiple moisture sensors in different positions within the housing, e.g., within different internal portions, the drug administration device can determine the location of moisture contamination. Additional moisture sensor(s) can be present beyond the first moisture sensor and the second moisture sensor.

The locations of the moisture sensors can include, for example, within the removable power supply such that moisture ingress that may affect the safety of the removable power supply can be detected, adjacent to the processor such that moisture ingress that may damage the processor and therefore the electrical systems within the drug administration device can be detected, adjacent to the motor, adjacent to the dispensing mechanism, adjacent to and outside of the drug holder, or within the drug holder.

The first moisture sensor and the second moisture sensor, and each of any additional moisture sensors, can be located in areas of the drug administration device that are delimited from each other such that a contaminant in one area cannot freely move to the other area, e.g., there is some impediment to the flow of the contaminant between the areas such as by different internal portions being sealed from each other. This location selection may provide an efficient use of the moisture sensors, since the moisture sensors will be measuring distinct regions of the drug administration device.

The processor can be configured to modify the operation of the drug administration device in a different way in response to different locations of moisture contamination within the housing. By adapting the response to moisture contamination to the location of the moisture contamination, the processor can be configured to modify the operation of the device to limit the functionality in relation to only the components of the drug administration device that are directly affected by the moisture contamination due to their location within the housing.

The housing can include a conductive trace, and the at least one sensor can include a sensor that is configured to measure a conductivity of the conductive trace, in particular the conductivity along the conductive trace. A deviation in the conductivity along the conductive trace can be indicative of a change in the integrity of the housing. The processor can be configured to detect the deviation using the measured conductivity from the sensor. The processor can be configured to modify the operation of the drug administration device, before contamination in the housing is detected, in response to damage in the housing as indicated by the detected deviation in the conductivity, as damage to the housing makes contamination within the housing more likely. The conductive trace can be positioned across a joint in the housing. This positioning allows monitoring of a location of the housing that can be particularly susceptible to damage and contaminant ingress.

The housing can include an electromagnetic shield configured to prevent electromagnetic radiation interacting with the drug administration device. By the housing including the electromagnetic shield, the risk of electromagnetic interference is reduced.

The electromagnetic shield can be present throughout the housing to provide complete protection for an interior of the housing. Alternatively, the electromagnetic shield can be present so as to protect only specific regions within the housing. These regions can be those areas with specific components that require protection from electromagnetic interference or are most likely to be adversely affected by electromagnetic interference.

The electromagnetic shield can be a passive electromagnetic shield or an active electromagnetic shield. A passive electromagnetic shield can include, e.g., sheet metal, a metal screen, or a metal foam. The passive electromagnetic shield can include a metallic ink or paint applied to the inside of the housing. The active electromagnetic shield can include, e.g., solenoids or Helmholtz coils configured to use static or low frequency electromagnetic shields to cancel out ambient electromagnetic field volume.

In another exemplary embodiment, a drug administration device configured to administer a drug includes a housing, a dispensing mechanism configured to dispense the drug and being disposed within the housing, a removable power supply configured to power the drug administration device and to be received by the housing, a first sensor configured to measure a parameter indicative of a remaining charge of the removable power supply and configured to output data, and a processor configured to receive data from the first sensor and to modify operation of the drug administration device in response to the data received from the first sensor.

The drug administration device can be any form of drug administration devices described herein.

The housing of the drug administration device is the external portion of the drug administration device. The housing of the drug administration device can be configured to keep contaminants out of an internal portion of the drug administration device, where the internal portion of the drug administration device includes all parts of the drug administration device disposed within the housing. In particular, the housing may be configured to be water resistant, e.g., waterproof to IPX2, IPX3, IPX4, IPX5, IPX6, IPX7, IPX8 or more, according to the IEC standard 60529. The housing can combine with a portion of the removable power supply when the removable power supply is received within the housing in order to provide an external portion of the drug administration device and, if the housing is configured to be water resistant or waterproof, the required water resistance or water proofing.

The drug holder of the drug administration device can be of any suitable form for holding the drug. As noted herein, such suitable forms include a vial or a syringe.

The removable power supply of the drug administration device can be a primary battery that is configured to be used once. Alternatively, the removable power supply can be a secondary battery that is configured to be recharged. The primary battery can include, e.g., a zinc-carbon battery or an alkaline battery. The secondary battery can include, e.g., a nickel-cadmium cell, a nickel-zinc cell, a nickel metal hydride cell, or a lithium-ion cell.

The first sensor is utilised to determine the remaining charge of the removable power supply (e.g., its state of charge) and output data, e.g., to the processor, indicative of the remaining charge. The first sensor can therefore be any sensor that can measure a parameter that is indicative of the state of charge of the removable power supply. One approach is to utilise a sensor configured to measure a voltage of the removable power supply and convert the measured voltage to a state of charge of the removable power supply via a look-up table that relates the measured voltage to the state of charge. Such a look-up table can be stored in a memory of the drug administration device. Another approach utilises a sensor configured to measure a current supplied by the removable power supply and relate the measured current to the expected charge remaining in the removable power supply. This is known as coulomb counting. A processor and a memory of the drug administration device can be utilised to perform this calculation to determine the expected state of charge.

The housing can be configured to receive a second power supply configured to power the drug administration device. This second power supply can be configured to provide power to the drug administration device in the absence of power from the removable power supply. By allowing a second power supply to be used when there is an absence of power from the removable power supply, the drug administration device can be used by a user while the removable power supply is being replaced or recharged. This redundancy of power supply avoids the user missing dosages of the drug or administering dosages late because the removable power supply of the drug administration device does not have enough power.

The drug administration device can include a second sensor configured to measure a parameter indicative of a remaining charge of the second power supply (its state of charge), and the processor can be configured to receive data from the second sensor and modify the operation of the drug administration device in response to the data received from the second sensor, which may allow the drug administration device and the user of the drug administration device to adapt usage of the device in response to not only the remaining charge of the removable power supply but also the remaining charge of the second power supply. The second sensor can have any of the features associated with the first sensor described herein, especially in relation to assessing the state of charge of the second power supply.

The second power supply can be a primary battery. The primary battery can be, e.g., a zinc-carbon battery or an alkaline battery. The primary battery can be a commercially available primary battery with a standard size such as an AA battery, an AAA battery, a C battery, an E battery, or a D battery. Utilising standard size batteries may allow the user to easily find replacements for the second power supply.

Alternatively, the second power supply can be a secondary battery, which allows the second power supply to be recharged and so avoids the need for the secondary battery to be replaced.

The drug administration device can include the second power supply. The second power supply can be disposed within the housing of the drug administration device.

The removable power supply and/or the second power supply can be configured to be wirelessly charged, e.g., charged without being connected physically to a power supply. An example of wireless charging is inductive charging. The wireless charging can utilise the Qi standard or the PMA standard. By configuring power supplies to be wirelessly charged, the drug administration device may not require an external socket to charge. The ability to charge wirelessly may reduce the time that the drug administration device needs to be physically connected to a power supply for charging. Wireless charging may also reduce the chances of contamination because, inter alia, there is one fewer port into the drug administration device and a reduction in user handling.

The second power supply can be configured to receive charge from the removable power supply when the removable power supply is received within the housing. By charging the second power supply from the removable power supply, the second power supply can be charged automatically. This eliminates the risk of the user forgetting to charge the second power supply before the removable power supply runs out of charge. Therefore, this reduces the risk of the drug administration device running out of charge.

The second power supply can be configured to power the drug administration device for a predetermined period of time, and the removable power supply can be configured to be chargeable from no charge (fully discharged) to full charge (fully charged) within the predetermined period of time. This configuration of the removable and second power supplies may allow the user to be able to maintain constant use of the drug administration device with only one removable power supply, thereby reducing the complexity of the drug administration device and improving compliance.

Modifying the operation of the drug administration device can include the processor being configured to initiate an alert when the remaining charge of the removable power supply reaches a first threshold level. The first threshold level can be enough charge for the dispensing mechanism to dispense five more dosages of the drug. The first threshold level can be enough charge for the dispensing mechanism to dispense four more dosages of the drug. The first threshold level can be enough charge for the dispensing mechanism to dispense three more dosages of the drug. The first threshold level can be enough charge for the dispensing mechanism to dispense two more dosages of the drug. The first threshold level can be enough charge for the dispensing mechanism to dispense one more dosage of the drug. The processor can be configured to initiate an alert via a device indicator and/or user interface of the drug administration device. The processor can be configured to initiate an alert by communicating, e.g., via a communications interface of the drug administration device, with another wireless device that has a user interface, for example a smartphone owned by or otherwise associated with the user. The alert allows the user to take appropriate action to address the low power charge level before the drug administration device runs out of charge.

The processor can be configured to initiate an alert when the remaining charge of the second power supply reaches a second threshold level. The second threshold level can be enough charge for the dispensing mechanism to dispense five more dosages of the drug. The second threshold level can be enough charge for the dispensing mechanism to dispense four more dosages of the drug. The second threshold level can be enough charge for the dispensing mechanism to dispense three more dosages of the drug. The second threshold level can be enough charge for the dispensing mechanism to dispense two more dosages of the drug. The second threshold level can be enough charge for the dispensing mechanism to dispense one more dosage of the drug. The processor can be configured to initiate an alert via a device indicator and/or user interface of the drug administration device. The processor can be configured to initiate an alert by communicating, e.g., via a communications interface of the drug administration device, with another wireless device that has a user interface, for example a smartphone owned by or otherwise associated with the user. The alert allows the user to take appropriate action to address the low power charge level before the drug administration device runs out of charge.

The removable power supply can include removable power supply identity data. The processor can be configured to receive the removable power supply identity data when the removable power supply is received within the housing. The processor can be configured to modify the operation of the drug administration device in response to the removable power supply identity data, e.g., in response to the removable power supply identity data not matching power supply data that identifies power supplies compatible with the drug administration device and that is stored in a memory of the drug administration device. The removable power supply identity data can be present on the removable power supply as part of a data storage component. The data storage component can be of the form of an integrated circuit that can communicate with the processor. The data storage component can be an radio frequency identification (RFID) tag. The data storage component can be in the form of a bar code. The removable power supply identity data can be stored on the removable power supply utilising a plurality of different forms of data storage components. By having a removable power supply that includes removable power supply data, the drug administration device may avoid the risk of damage resulting from incompatible power inputs.

The drug administration device can include a suitable component for acquiring the removable power supply identity data. For example, when the removable power supply identity data is contained as part of an RFID tag, acquiring the removable power supply identity data involves the use of an RFID scanner. A bar code scanner can be utilised when the removable power supply identity data is contained within the form of a bar code. When the removable power supply identity data is in the form of an integrated circuit, the integrated circuit can be powered and capable of transmitting the removable power supply identity data. Therefore, acquiring the removable power supply identity data requires the use of an appropriate communications interface for receiving the removable power supply identity data communicated from the removable power supply identity data.

The removable power supply can include a memory configured to receive data. This data can be usage data from the drug administration device, e.g., amounts and timing of dosages administered by the drug administration device. Where the removable power supply has a longer lifespan than the other components of the drug administration device, the memory configured to receive data can allow a digital continuity to a drug administration regimen administered by the drug administration device. This memory can include flash memory, one or more varieties of random access memory (RAM) (e.g. static RAM (SRAM), dynamic RAM (DRAM), or synchronous DRAM (SDRAM)), and/or a combination of memory technologies.

The removable power supply can include a suitable communications interface for receiving data from other component(s) of the drug administration device, such as data originating from the processor. Data can be communicated to the removable power supply from the other component(s) of the drug administration device via, e.g., direct electrical contacts when the removable power supply is present as part of the drug administration device.

The dispensing mechanism can be configured to be powered by the removable power supply. The dispensing mechanism can be powered by the removable power supply via an electric motor. For example, when the drug holder is a syringe, the removable power supply can power the electric motor that powers a driver of the drug administration device for a plunger of the drug administration device that expels the drug from the drug holder.

The dispensing mechanism can include a device operation prevention mechanism. The device operation prevention mechanism can be configured to prevent the dispensing mechanism from operating. The device operation prevention mechanism can prevent operation of the dispensing mechanism by preventing the dispensing mechanism from being powered by the removable power supply. The device operation prevention mechanism can be configured to be enabled when the removable power supply identity data indicates that the removable power supply is not compatible with the drug administration device, e.g., the processor can be configured to enable the device operation prevention mechanism when the processor determines using the removable power supply identity data that the removable power supply is compatible with the drug administration device.

The device operation prevention mechanism can be configured to prevent the dispensing mechanism from being powered by the removable power supply when the remaining charge in the removable power supply falls below a third threshold level. The third threshold level can be enough power for the dispensing mechanism to complete a full drug administration sequence. A full drug administration sequence is a drug administration sequence in which a complete dose of the drug is administered. This prevention may ensure that the drug administration device does not administer a partial dosage of the drug that would result in the patient deviating from their dosage regimen. Where the dispensing mechanism and/or other part(s) of the drug administration device undergoes mechanical movement during a drug administration sequence, e.g., where a drug administration sequence includes the extension of a needle outside of the housing of the drug administration device, then a full drug administration sequence can also include the dispensing mechanism returning to its initial configuration.

The drug dispensing mechanism can be configured to be operated manually and/or automatically. The automatic operation requires power, such as from the removable power supply, in order to be effected. The drug dispensing mechanism can be operated manually by the drug administration device including a pump or plunger that is accessible to a user of the drug administration device. The pump or plunger can be configured to drive a defined amount of drug from the drug holder. The pump or plunger can be spring loaded to ensure complete administration of a full dose of the drug. By allowing the user to manually operate the drug dispensing mechanism, the drug administration device can be used even when it runs out of power.

The device operation prevention mechanism can be configured to prevent the dispensing mechanism from being operated manually when the remaining charge of the removable power supply is above a fourth threshold level. This prevention may stop the user from administering further dosages beyond what is prescribed when the drug administration device has remaining charge after all prescribed dosages have been delivered from the drug administration device. This functionality is particularly suited to drug administration devices where the drug administration device is configured to administer a drug that is prone to abuse.

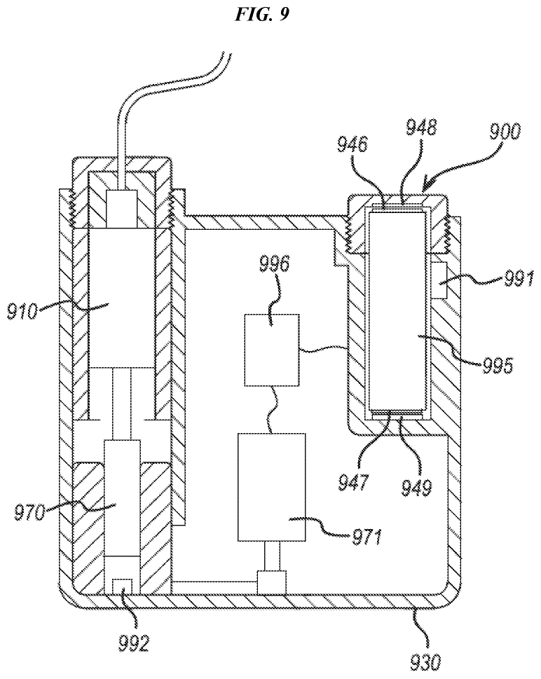

FIG. 9 depicts an embodiment of a drug administration device 900 with multiple sensors. The drug administration device 900 includes a removable power supply 995 configured to supply power to other components of the drug administration device 900. The removable power supply 995 includes a first power supply electrical contact 946 and a second power supply electrical contact 947. When the removable power supply 995 is received within the drug administration device's housing 930, as shown in FIG. 9, the first and second power supply electrical contacts 946, 947 are touching a first housing electrical contact 948 and a second housing electrical contact 949, respectively. The removable power supply 995 is configured to supply power to the drug administration device's processor 996. The processor 996 is configured to control the drug administration device's motor 971, which is configured to power the drug administration device's dispensing mechanism 970. The dispensing mechanism 970 is a driver for a plunger that drives the drug out of the drug administration device's drug holder 910. The drug holder 910 is a syringe.

The first and second power supply electrical contacts 946, 947 and the first and second housing electrical contacts 948, 949 are configured to complete a circuit between the power supply 995 and the rest of the drug administration device to which the power supply 995 is configured to supply power when the power supply 995 is received within the housing 930 of the drug administration device 900. The first and second power supply electrical contacts 946, 947 and the first and second housing electrical contacts 948, 949 can be made out of a metallic material. The first power supply electrical contact 946 can be a positive terminal of the power supply 995, and the second power supply electrical contact 947 can be a negative terminal of the power supply 995. The first housing electrical contact 948 can be configured to receive the positive terminal 946 of the power supply 995, and the second housing electrical contact 949 can be configured to receive the negative terminal 947 of the power supply 995.

The drug administration device 900 includes a first moisture sensor 991 located by the drug administration device's removable power supply compartment, and thus by the removable power supply 995 when the power supply 995 is received in the removable power supply compartment, and a second moisture sensor 992 located by the dispensing mechanism 970. The first moisture sensor 991 and the second moisture sensor 992 are thus located in delimited regions where a flow of contaminants is inhibited between these regions. Moisture sensors suitable for use as the first and second moisture sensors 991, 992 include hygrometers and/or water detectors. Examples of hygrometers include capacitive hygrometers (which measure the dielectric constant of a polymer or metal oxide, which varies with humidity), resistive hygrometers (which measure the electrical resistance of a material, which varies with humidity), thermal hygrometers (which measure the change in thermal conductivity of air due to humidity), gravimetric hygrometers (which measure the mass of an air sample compared to an equal volume of dry air), and optical hygrometers (which measure the absorption of light by water in the air). Water detectors can include two electrical contacts disposed near to each other and a resistance meter configured to measure the resistance between the two contacts such that when water bridges the two contacts, a decrease in resistance is measured and a processor (e.g., the processor 996) can identify that water is present.

The processor 996 is configured to receive data from the first moisture sensor 991 and the second moisture sensor 992. From this data, the processor 996 can be configured to determine the location of any moisture contamination. This determination allows the processor 996 to modify the operation of the drug administration device 900 in response to the location of moisture contamination.

FIG. 10a is a schematic representation of an embodiment of housing and power supply electrical contacts. A housing 1030 includes a first housing electrical contact 1031 and a second housing electrical contact 1032. The first housing electrical contact 1031 and the second housing electrical contact 1032 are protected by a housing protection mechanism 1033, which is in its first configuration. The housing protection mechanism 1033 is a membrane that seals the first housing electrical contact 1031 and the second housing electrical contact 1032. The membrane can be an impermeable membrane. The membrane can be made of an elastomeric material. The membrane can include silicone. The membrane can include a rubber. A power supply 1095 includes a first power supply electrical contact 1036 and a second power supply electrical contact 1037. The first power supply electrical contact 1036 and the second power supply electrical contact 1037 are configured to pierce the housing protection mechanism 1033. The first power supply electrical contact 1036 and the second power supply electrical contact 1037 can be configured to pierce the housing protection mechanism 1033 by having a sharp element or feature. The housing protection mechanism 1033 can be configured to be pierced. The housing protection mechanism 103 can include pre-formed openings or localised structural weaknesses configured to be pierced by the first and second power supply electrical contacts. The first housing electrical contact 1031 is configured to have a complementary shape to the first power supply electrical contact 1036, and the second housing electrical contact 1032 is configured to have a complementary shape to the second power supply electrical contact 1037, thus providing sufficient contact area for the transfer of electrical current. The first and second power supply electrical contacts 1036, 1037 and the first and second housing contacts 1031, 1032 can be configured to complete a circuit between the power supply 1095 and the rest of the drug administration device to which the power supply 1095 is configured to supply power when the power supply 1095 is received within the housing 1030 of the drug administration device. The first and second power supply electrical contacts 1036, 1037 and the first and second housing contacts 1031, 1032 can be made out of a metallic material.

FIG. 10b is a schematic representation of the housing and power supply electrical contacts 1031, 1032, 1036, 1037 of the embodiment of FIG. 10a. The housing protection mechanism 1033 is in its second configuration. The first power supply electrical contact 1036 and the second power supply electrical contact 1037 have pierced the housing protection mechanism 1033 to allow the first power supply electrical contact 1036 to contact the first housing electrical contact 1031 and to allow the second power supply electrical contact 1037 to contact the second housing electrical contact 1032. The drug administration device of this embodiment goes from the first configuration represented in FIG. 10a to the second configuration represented in FIG. 10b when the removable power supply 1095 is inserted into the housing 1030. By inserting the removable power supply 1095 into the housing 1030, the first power supply electrical contact 1036 and the second power supply electrical contact 1037 each pierce the housing protection mechanism 1033 and displace membrane material, thus putting the housing protection mechanism 1033 into its second configuration. The membrane 1033 is elastic and so returns from its second position (FIG. 10b) to its first position (FIG. 10a) when the removable power supply 1095 is subsequently removed from the housing 1030. The membrane 1033 can include a preformed hole configured to receive the first and/or second power supply electrical contact. The first and second power supply electrical contacts 1036, 1037 can be configured to pierce the membrane by each cutting a hole in the membrane 1033 when the removable power supply 1095 is received within the housing 1030 of the drug administration device for the first time. The first and second power supply electrical contacts 1036, 1037 can pierce the membrane 1033 via the holes formed in the membrane 1033 by the first and second power supply electrical contacts 1036, 1037 when subsequently received within the housing 1030 of the drug administration device. The membrane 1033 can extend over the entirety of the housing 1030.

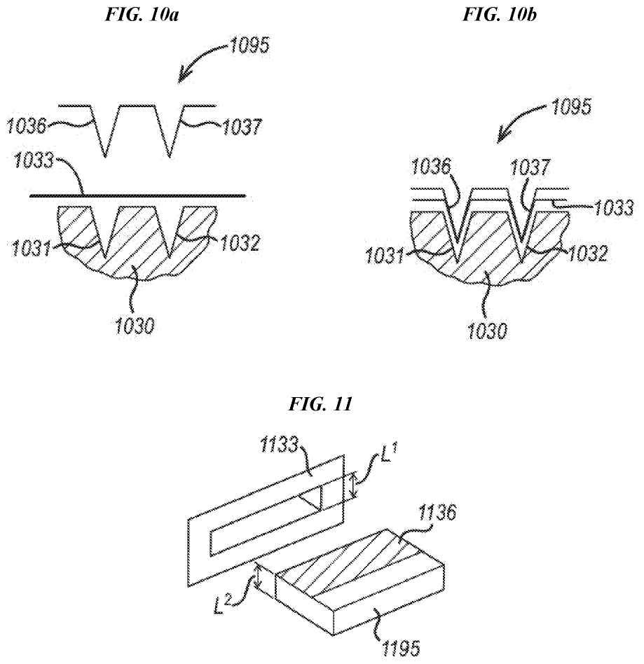

FIG. 11 depicts an embodiment of a housing protection mechanism 1133 in its first configuration and a first power supply electrical contact 1136. The housing protection mechanism 1133 includes a hole in the form of a slit of width L₁, and the first power supply electrical contact 1136 is disposed on a portion of the removable power supply 1195 that has a width L₂. The width L₂ is greater than the width L₁. The housing protection mechanism 1133 is made of an elastomeric material so that it can be pushed into its second configuration by the insertion of the first power supply electrical contact 1136. The elastomeric material can be an elastomer. The elastomeric material can be a rubber. The elastomeric material can be a saturated rubber. The elastomeric material can be an unsaturated rubber. The elastomeric material can be natural rubber. Examples of the elastomeric material include neoprene rubber, natural polyisoprene, polybutadiene, chloroprene, butyl rubber, styrene-butadiene, nitrile rubber, ethyl propylene rubber, epichlorohydrin rubber, polyacrylic rubber, silicone rubber, fluorosilicone, fluoroelastomers, perfluoroelastomers, polyether block amides, chlorosulfonated polyethylene, and ethylene-vinyl acetate. In this way, the first configuration of the housing protection mechanism has a smaller opening (slit) than the second configuration. This smaller size of the slit in the first configuration impedes the ingress of contaminants towards the housing's first electrical contact.

The first power supply electrical contact 1136 is configured to pass through the slit of the housing protection mechanism 1133 when the removable power supply 1195 is received within the housing 1030. As the width L₂ is greater than the width L₁, the housing protection mechanism 1133 is in a stressed second positon when the removable power supply 1195 is received within the housing. The edge of the slit of the housing protection mechanism 1133 moves across a surface of the first power supply electrical contact 1136 when going from its first configuration to its second configuration. Therefore, the surface of the first power supply electrical contact 1136 is wiped by the housing protection mechanism 1133 when the removable power supply 1195 is inserted into the housing of the drug administration device. The removal of the first power supply electrical contact 1136 from the housing protection mechanism 1133 allows the housing protection mechanism 1133 to revert to the smaller width L₁.

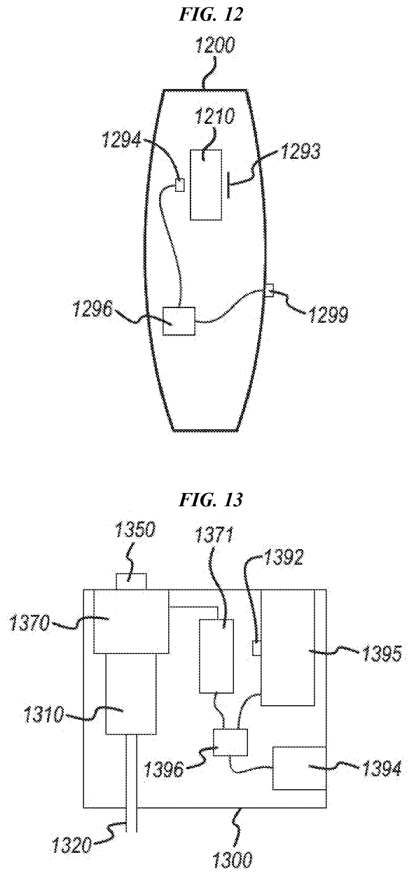

FIG. 12 depicts an embodiment of a drug administration device 1200 including a system configured to detect contamination within the drug administration device's drug holder 1210. The drug administration device 1200 includes a photosensor 1294 positioned on one side of the drug holder 1210, and the drug administration device 1200 includes a light source 1293 positioned on the opposite side of the drug holder 1210 and facing the photosensor 1294. The photosensor 1294 is connected by a wire to a processor 1296. The processor 1296 is configured to receive data from the photosensor 1294 and to determine from this data whether or not the drug holder 1210 contains a contaminant. The processor 1296 can be configured to determine whether the drug holder 1210 contains a contaminant by comparing the data provided by the photosensor 1294 to a threshold band, e.g., as stored in a memory of the drug administration device 1200. If the data provided by the photosensor 1294 falls outside of the threshold band then the drug holder 1210 can be determined to contain a contaminant. The drug administration device 1200 also includes an LED 1299. The processor 1296 is configured to communicate with the LED 1299 and is configured to turn the LED 1299 on when there is a contaminant in the drug holder 1210. This lighting of the LED 1299 warns the user to not use the drug administration device 1200 and prompts the user to replace the drug in the drug holder 1210, the drug administration device 1200, and/or the drug holder 1210.

FIG. 13 depicts an embodiment of a drug administration device 1300 configured to be operable both automatically and manually. The drug administration device 1300 is an infusion pump. The infusion pump 1300 includes a drug holder 1310, a dispensing mechanism 1370, and a drug outlet 1320. The drug holder 1310 is a syringe including a vial and a plunger. The dispensing mechanism 1370 is configured to be operated by a manual pump 1350 or a motor 1371. The dispensing mechanism 1370 is configured to drive the plunger in the drug holder 1310, pushing the drug out of the drug outlet 1320. The infusion pump 1300 includes a removable power supply 1395 and a second power supply 1394 configured to provide power to the electronic components of the infusion pump 1300. The infusion pump 1300 also includes a sensor 1392 configured to measure a remaining charge of the removable power supply 1395. The infusion pump 1300 includes a processor 1396 which is configured to receive data from the sensor 1392 and is configured to modify the operation of the infusion pump 1300 in response to this data. If the data indicates that the removable power supply 1395 does not contain enough charge to power the dispensing mechanism 1370 for a full drug administration sequence, then the processor 1396 is configured to stop the motor 1371 from powering the dispensing mechanism 1370. In such situations, the dispensing mechanism 1370 can be operated by the manual pump 1350. The manual pump 1350 is configured to only allow a full dose of the drug to be administered. This allows the infusion pump 1300 to be used even when the removable power supply 1395 runs out of charge.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 published Aug. 13, 2009 and entitled “System And Method Of Sterilizing An Implantable Medical Device.” It is preferred that device, if implanted, is hermetically sealed. This can be done by any number of ways known to those skilled in the art.

The present disclosure has been described above by way of example only within the context of the overall disclosure provided herein. It will be appreciated that modifications within the spirit and scope of the claims may be made without departing from the overall scope of the present disclosure.

What is claimed is:

1. A drug administration device configured to administer a drug comprising:
a housing, wherein the housing comprises a first housing electrical contact and a second housing electrical contact;
a dispensing mechanism configured to dispense the drug, wherein the dispensing mechanism is disposed within the housing;
a drug holder configured to hold the drug; and
a removable power supply configured to power the drug administration device, wherein the removable power supply comprises a first power supply electrical contact and a second power supply electrical contact; and
wherein
the housing comprises a housing protection mechanism configured to be in
a first configuration configured to protect the first housing electrical contact when the removable power supply is not received within the housing; and
a second configuration configured to enable contact between the first housing electrical contact and the first power supply electrical contact and between the second housing electrical contact and the second power supply electrical contact when the removable power supply is received within the housing;
and/or
the removable power supply comprises a power supply protection mechanism configured to be in
a first configuration configured to protect the second power supply electrical contact when the removable power supply is not received in the housing; and
a second configuration configured to enable contact between the first housing electrical contact and the first power supply electrical contract and between the second housing electrical contact and the second power supply electrical contact when the removable power supply is received within the housing.

2. The drug administration device of claim 1, wherein the housing comprises the housing protection mechanism.

3. The drug administration device of claim 1 or claim 2, wherein the removable power supply comprises the removable power supply protection mechanism.

4. The drug administration device of any preceding claim, wherein the housing protection mechanism in the first configuration forms a seal around the first housing electrical contact.

5. The drug administration device of claim 4, wherein the housing protection mechanism in the first configuration forms a seal around the second housing electrical contact.

6. The drug administration device of claim 4, wherein the housing protection mechanism comprises a membrane, and the first power supply electrical contact power supply is configured to pierce the membrane such that the membrane is in the second configuration when the removable power supply is received within the housing.

7. The drug administration device of claim 5, wherein the first power supply electrical contact and the second power supply electrical contact are each configured to pierce the membrane such that the membrane is in the second configuration when the removable power supply is received within the housing.

8. The drug administration device of any one of claims 1 to 3, wherein the power supply protection mechanism in the first configuration forms a seal around the second power supply electrical contact.

9. The drug administration device of claim 8, wherein the power supply protection mechanism in the first configuration forms a seal around the first power supply electrical contact.

10. The drug administration device of claim 8, wherein the power supply protection mechanism is a membrane, and the second housing electrical contact is configured to pierce the membrane such that the membrane is in the second configuration when the removable power supply is received within the housing.

11. The drug administration device of claim 9, wherein the first housing electrical contact is configured to pierce the membrane such that the membrane is in the second configuration when the removable power supply is received within the housing.

12. The drug administration device of any one of claims 1 to 3, wherein the housing protection mechanism in the first configuration forms a seal around the first housing electrical contact, and wherein the power supply protection mechanism in the first configuration forms a seal around the second power supply electrical contact.

13. The drug administration device of claim 12, wherein each of the housing protection mechanism and the power supply protection mechanism comprises a membrane, and the first power supply electrical contact and the second housing electrical contact are each configured to pierce the respective membranes such that each membrane is in the second configuration when the removable power supply is received within the housing.

14. The drug administration device of any preceding claim, wherein the housing protection mechanism is configured to move from the first configuration to the second configuration by being contacted by the removable power supply when the removable power supply is inserted into the housing.

15. The drug administration device of claim 14, wherein the housing protection mechanism is configured to move along a surface of the first housing electrical contact.

16. The drug administration device of claim 15, wherein the housing protection mechanism is configured to move along a surface of the second housing electrical contact when the housing protection mechanism moves from the first configuration to the second configuration.

17. The drug administration device of any preceding claim, wherein the power supply protection mechanism is configured to move from the first configuration to the second configuration by being contacted by the housing when the removable power supply is inserted into the housing.

18. The drug administration device of claim 17, wherein the power supply protection mechanism is configured to move along a surface of the first power supply electrical contact.

19. The drug administration device of claim 18, wherein the power supply protection mechanism is configured to move along a surface of the second power supply electrical contact when the power supply protection mechanism moves from the first configuration to the second configuration.

20. The drug administration device of any preceding claim, wherein at least one of the first housing electrical contact, the second housing electrical contact, the first power supply electrical contact, and the second power supply electrical contact is coated with a hydrophobic conductive coating.

21. The drug administration device of any preceding claim, further comprising at least one sensor located within the housing configured to detect an environmental parameter within the housing; and a processor, wherein the processor is configured to receive data from the at least one sensor and to modify the operation of the drug administration device in response to the data received from the at least one sensor.

22. The drug administration device of claim 21, wherein the at least one sensor comprises a moisture sensor.

23. The drug administration device of claim 21 or claim 22, wherein the at least one sensor comprises a temperature sensor.

24. The drug administration device of any preceding claim, wherein the drug comprises at least one of infliximab, golimumab, ustekinumab, daratumumab, guselkumab, epoetin alfa, risperidone, and paliperidone palmitate.

25. A drug administration device configured to administer a drug comprising:
a housing;
a dispensing mechanism configured to dispense the drug, wherein the dispensing mechanism is disposed within the housing;
a drug holder configured to hold the drug;
at least one sensor located within the housing configured to detect an environmental parameter within the housing; and
a processor, wherein the processor is configured to receive data from the at least one sensor and to modify the operation of the drug administration device in response to the data received from the at least one sensor.

26. The drug administration device of any one of claims 21 to 25, wherein the at least one sensor comprises at least one contamination sensor.

27. The drug administration device of claim 26, wherein the at least one contamination sensor comprises a moisture sensor or a photosensor.

28. The drug administration device of claim 27, wherein the at least one contamination sensor comprises a photosensor, the photosensor is configured to detect light from the drug holder, and the processor is configured to determine whether the drug holder contains a contaminant based on the data received from the photosensor.

29. The drug administration device of claim 28, wherein the drug holder is transparent to at least a wavelength of light that the photosensor is configured to detect.

30. The drug administration device of claim 28 or claim 29, further comprising a light source configured to irradiate the drug holder, wherein the photosensor is configured to detect the light from the light source after it has passed through the drug holder.

31. The drug administration device of any one of claims 21 to 30, wherein the processor is configured to modify the operation of the drug administration device in response to the data received from the at least one sensor by:
beginning a limp mode operation, wherein the limp mode operation comprises reduced functionality, when a contaminant is determined to be present;
indicating to a user the status of the drug administration device;
preventing startup of the drug administration device, when a contaminant is determined to be present; and/or
communicating with a remote server, when a contaminant is determined to be present.

32. The drug administration device of any one of claims 21 to 31, wherein the at least one sensor comprises a moisture sensor and wherein the processor is configured to modify the operation of the drug administration device when the processor receives data from the moisture sensor indicating that moisture contamination has been detected within the housing.

33. The drug administration device of any one of claims 21 to 32, wherein the at least one sensor comprises a first moisture sensor located at a first position in the housing and a second moisture sensor located at a second position within the housing.

34. The drug administration device of claim 33, wherein the processor is configured to receive data from the first moisture sensor and the second moisture sensor and to determine a location of moisture contamination within the housing from the data.

35. The drug administration device of claim 34, wherein the processor is configured to modify the operation of the drug administration device in a different way in response to different locations of moisture contamination within the housing.

36. The drug administration device of any preceding claim, wherein the housing comprises a conductive trace, and wherein the at least one sensor comprises a sensor that is configured to measure a conductivity of the conductive trace.

37. The drug administration device of claim 36, wherein the conductive trace is positioned across a join in the housing.

38. The drug administration device of any preceding claim, wherein the housing comprises an electromagnetic shield configured to prevent electromagnetic radiation interacting with the drug administration device.

39. The drug administration device of any preceding claim, wherein the drug comprises at least one of infliximab, golimumab, ustekinumab, daratumumab, guselkumab, epoetin alfa, risperidone, and paliperidone palmitate.

40. A drug administration device configured to administer a drug comprising:
a housing;
a dispensing mechanism configured to dispense the drug, wherein the dispensing mechanism is disposed within the housing;
a removable power supply configured to power the drug administration device, wherein the housing is configured to receive the removable power supply;
a first sensor, wherein the first sensor is configured to measure a parameter indicative of the remaining charge of the removable power supply and output data; and
a processor, wherein the processor is configured to receive the data from the first sensor and modify the operation of the drug administration device in response to the data received from the first sensor.

41. The drug administration device of claim 40, wherein the housing is configured to receive a second power supply configured to power the drug administration device.

42. The drug administration device of claim 41, further comprising a second sensor, wherein the second sensor is configured to determine the remaining charge of the second power supply, and wherein the processor is configured to receive data from the second sensor and modify the operation of the drug administration device in response to the data received from the second sensor.

43. The drug administration device of claim 41 or claim 42, wherein the second power supply is a primary cell.

44. The drug administration device of claim 41 or claim 42, wherein the drug administration device comprises the second power supply, wherein the second power supply is disposed within the housing.

45. The drug administration device of claim 44, wherein the second power supply is configured to be wirelessly charged.

46. The drug administration device of claim 44 or claim 45, wherein the drug administration device is configured to charge the second power supply using the removable power supply when the removable power supply is received within the housing.

47. The drug administration device of any one of claims 44 to 46, wherein the second power supply is configured to power the drug administration device for a predetermined period of time, wherein the removable power supply is configured to be chargeable from no charge to full charge within the predetermined period of time.

48. The drug administration device of any one of claims 40 to 47, wherein modifying the operation of the drug administration device comprises the processor being configured to initiate an alert when the remaining charge of the removable power supply reaches a first threshold level.

49. The drug administration device of any one of claims 40 to 48, wherein the processor is configured to initiate an alert when the remaining charge of the second power supply reaches a second threshold level.

50. The drug administration device of any one of claims 40 to 49, wherein the removable power supply comprises removable power supply identity data, and the processor is configured to receive the removable power supply identity data when the removable power supply is received within the housing, and wherein the processor is configured to modify the operation of the drug administration device in response to the removable power supply identity data.

51. The drug administration device of any one of claims 40 to 50, wherein the removable power supply further comprises a memory configured to receive data.

52. The drug administration device of any one of claims 40 to 51, wherein the dispensing mechanism is configured to be powered by the removable power supply.

53. The drug administration device of any one of claims 40 to 52, wherein the dispensing mechanism comprises a device operation prevention mechanism.

54. The drug administration device of claim 53 when directly or indirectly dependent on claim 50, wherein modifying the operation of the drug administration device comprises enabling the device operation prevention mechanism to prevent the dispensing mechanism from being powered by the removable power supply when the removable power supply identity data indicates that the removable power supply is not compatible with the drug administration device.

55. The drug administration device of claim 53 or claim 54, wherein the device operation prevention mechanism is configured to prevent the dispensing mechanism from being powered by the removable power supply when the remaining charge in the removable power supply falls below a third threshold level.

56. The drug administration device of claim 55, wherein the third threshold level is enough charge for the dispensing mechanism to complete a full drug administration sequence.

57. The drug administration device of any one of claims 40 to 56, wherein the drug dispensing mechanism is configured to be operated manually.

58. The drug administration device of claim 57 when directly or indirectly dependent on claim 53, wherein the device operation prevention mechanism is configured to prevent the dispensing mechanism from being operated manually when the remaining charge of the removable power supply is above a fourth threshold level.

59. The drug administration device of any one of claims 40 to 59, wherein the drug comprises at least one of infliximab, golimumab, ustekinumab, daratumumab, guselkumab, epoetin alfa, risperidone, and paliperidone palmitate.
